(19) United States
(12) Patent Application Publication
(10) Pub. No.: US 2003/0078424 A1
(43) Pub. Date: Apr. 24, 2003

(54) Method for oxidizing tertiary amines and aromatic heterocycles containing nitrogen

(76) Inventors: Hanns Wurziger, Darmstadt (DE); Guido Pieper, Darmstadt (DE); Norbert Schwesinger, Eching (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/258,479

(22) PCT Filed: Apr. 5, 2001
(86) PCT No.: PCT/EP01/03873

(30) Foreign Application Priority Data
Apr. 27, 2000 (DE) 100 20 630.1

(51) Int. Cl. C07D 213/89; C 07D 2 7/46

(52) U.S. Cl. 546/1; 548/541; 564/298

(57) ABSTRACT

The present invention relates to a process for the oxidation of tertiary amines and nitrogen-containing aromatic heterocycles to amine oxides.

The present invention relates to a process for the oxidation of tertiary amines and nitrogen-containing aromatic heterocycles to amine oxides.

The oxidation of tertiary amines and nitrogen-containing aromatic heterocycles is a very common process in the chemical industry and its great importance is also reflected in numerous publications on this subject.

However, such oxidations carried out on the industrial scale have safety problems and dangers associated with them. On the one hand, relatively large quantities of highly toxic chemicals are frequently used, which in themselves already represent a considerable risk for man and the environment, and on the other hand, these oxidation processes are often very highly exothermic, creating an increased explosion hazard when these reactions are carried out on the industrial scale. Obtaining official approval, under the terms of BimschG (German Air-borne Pollution Act), to operate industrial plants for the oxidation of tertiary amines and nitrogen-containing aromatic compounds therefore involves considerable expenditure.

The object of the present invention is therefore to provide a process for the oxidation of tertiary amines and nitrogen-containing aromatic heterocycles to amine oxides which avoids the abovementioned disadvantages. In particular, it should be possible to carry out this process in a simple, reproducible manner with increased safety for man and the environment and with good yields, and the reaction conditions should be very controllable.

Surprisingly, this object is achieved by the process according to the invention for the oxidation of tertiary amines and/or nitrogen-containing aromatic heterocycles, wherein at least one tertiary amine and/or at least one nitrogen-containing aromatic heterocycle, in liquid or dissolved form, is mixed with at least one oxidizing agent, in liquid or dissolved form, in at least one microreactor, the mixture is reacted for a certain residence time and the amine oxide(s) formed is (are) optionally isolated from the reaction mixture.

Advantageous embodiments of the process according to the invention are described in the subclaims.

According to the invention, individual tertiary amines or nitrogen-containing aromatic heterocycles, or mixtures of at least two of these compounds, can be reacted by the process claimed, although it is preferred to use only one amine or only one nitrogen-containing aromatic heterocycle in the process according to the invention.

In terms of the invention, a microreactor is a reactor with a volume of ≦1000 μl in which the liquids and/or solutions are intimately mixed at least once. The volume of the microreactor is preferably ≦100 μl and particularly preferably ≦50 μl.

The microreactor is preferably made of thin interconnected silicon structures.

The microreactor is preferably a miniaturized continuous reactor and particularly preferably a static micromixer. Very particularly preferably, the microreactor is a static micromixer such as that described in the patent application with international publication number WO 96/30113, which is incorporated here by way of reference and constitutes part of the disclosure. Such a microreactor has small channels in which liquids and/or solutions of chemical compounds are mixed together by the kinetic energy of the flowing liquids and/or solutions.

The channels of the microreactor have a diameter preferably of 10 to 1000 μm, particularly preferably of 20 to 800 μm and very particularly preferably of 30 to 400 μm.

The liquids and/or solutions are pumped into the microreactor so as to flow through it at a rate preferably of 0.01 μl/min to 100 ml/min and particularly preferably of 1 μl/min to 1 ml/min.

According to the invention, the microreactor is preferably capable of being kept at a constant temperature.

According to the invention, the microreactor is preferably connected via an outlet to at least one detention section, preferably a capillary and particularly preferably a capillary capable of being kept at a constant temperature. After they have been thoroughly mixed in the microreactor, the liquids and/or solutions are transferred to this detention section or capillary to prolong their residence time.

In terms of the invention, the residence time is the time between the thorough mixing of the educts and the work-up of the resulting reaction solution for analysis or isolation of the desired product(s).

The necessary residence time in the process according to the invention depends on a variety of parameters, e.g. the temperature or the reactivity of the educts. Those skilled in the art will be able to adapt the residence time to these parameters and thereby optimize the course of the reaction.

The residence time of the reaction solution in the system used, consisting of at least one microreactor and optionally a detection section, can be adjusted by the choice of flow rate of the liquids and/or solutions used.

Another preferred procedure is to pass the reaction mixture through two or more microreactors connected in series. The result is that, even with an increased flow rate, the residence time is prolonged and the components used in the oxidation reaction are reacted so as to optimize the product yield of the amine oxide(s).

In another preferred embodiment, the reaction mixture is passed through two or more microreactors arranged in parallel in order to increase the throughput.

In another preferred embodiment of the process according to the invention, the number and arrangement of the channels in one or more microreactors are varied to prolong the residence time so that, here again, with an increased flow rate, the yield of the desired amine oxide(s) is optimized.

Preferably, the residence time of the reaction solution in the microreactor or, if appropriate, in the microreactor and the detention section is ≦15 hours, preferably ≦3 hours and particularly preferably ≦1 hour.

The process according to the invention can be carried out over a very wide temperature range which is limited essentially by the temperature resistance of the materials used to construct the microreactor and, if appropriate, the detention section, as well as other components, e.g. connectors and seals, and by the physical properties of the solutions and/or liquids used. Preferably, the process according to the invention is carried out at a temperature of −100 to +250° C., preferably of −78 to +150° C. and particularly preferably of 0 to +40° C.

The process according to the invention can be carried out continuously or batchwise, preferably continuously.

For carrying out the process according to the invention for the oxidation of tertiary amines and/or nitrogen-containing aromatic heterocycles, it is necessary for the oxidation to be carried out as far as possible in a homogeneous liquid phase containing no solid particles or only very small solid particles, as otherwise the channels in the microreactors become clogged.

The course of the oxidation reaction in the process according to the invention can be monitored by various analytical methods known to those skilled in the art, and optionally regulated. The course of the reaction is monitored preferably by chromatography and particularly preferably by high performance liquid chromatography, and optionally regulated. This markedly improves control of the reaction compared with known processes.

After the reaction, the amine oxides formed are optionally isolated. The amine oxide(s) is (are) preferably isolated from the reaction mixture by extraction.

Any of the tertiary amines known to those skilled in the art as substrates for oxidation to amine oxides can be used as tertiary amines in the process according to the invention. The tertiary amine used is preferably an aliphatic, cycloaliphatic, aromatic or heteroaromatic tertiary amine. The aliphatic, cycloaliphatic, aromatic or heteroaromatic radicals bonded to the nitrogen atom can be identical or different.

Any of the nitrogen-containing aromatic heterocycles known to those skilled in the art as substrates for oxidation to amine oxides can be used as nitrogen-containing aromatic heterocycles in the process according to the invention. The nitrogen-containing aromatic heterocycles used have a ring size preferably of 5 to 7 atoms and particularly preferably of 5 or 6 atoms. The nitrogen-containing aromatic heterocycle used is very particularly preferably pyridine and/or pyrimidine and/or pyrazine.

Nitrogen-containing aromatic heterocycles also include aromatic compounds and/or derivatives thereof which have at least one monocyclic and/or polycyclic nitrogen-containing aromatic parent structure or a corresponding partial structure, e.g. in the form of substituents. These nitrogen-containing aromatic parent structures or partial structures can also contain other heteroatoms, preferably oxygen and/or sulphur.

Any of the oxidizing agents known to those skilled in the art as suitable for the oxidation of tertiary amines and/or nitrogen-containing aromatic heterocycles to amine oxides, or a mixture of at least two of these oxidizing agents, can be used as oxidizing agents in the process according to the invention. It is preferred to use only one oxidizing agent.

The oxidizing agent is preferably at least one compound selected from inorganic and organic peroxides, hydrogen peroxide, mixtures of peroxo compounds with organic acids and/or inorganic acids and/or Lewis acids, organic per-acids, inorganic per-acids and dioxirans, or a mixture of at least two of these oxidizing agents.

The inorganic peroxide used is preferably an ammonium peroxide, an alkali metal peroxide, preferably sodium peroxide, an ammonium persulfate, an alkali metal persulfate, an ammonium perborate, an alkali metal perborate, an ammonium percarbonate, an alkali metal percarbonate, an alkaline earth metal peroxide, zinc peroxide or a mixture of at least two of these peroxides.

The organic peroxide used is preferably tert-butyl hydroperoxide, cumene hydroperoxide, menthyl hydroperoxide, 1-methylcyclohexane hydroperoxide or a mixture of at least two of these peroxides.

Potassium peroxodisulfate with sulfuric acid is used as the peroxo compound with an inorganic acid, and hydrogen peroxide with boron trifluoride is used as the peroxo compound with a Lewis acid.

Preferred organic per-acids are peroxybenzoic acid, m-chloroperoxybenzoic acid, p-nitroperoxybenzoic acid, magnesium monoperoxyphthalic acid, peroxyacetic acid, peroxymaleic acid or peroxytrifluoroacetic acid. It is also possible to use a mixture of at least two of these per-acids.

In the process according to the invention, the molar ratio of tertiary amine and/or nitrogen-containing aromatic heterocycle to oxidizing agent used depends on the reactivity of the amines and/or aromatic heterocycles used and of the oxidizing agents. The oxidizing agent and the tertiary amine or the nitrogen-containing aromatic heterocycle are preferably used in an equimolar ratio. In another preferred embodiment, the oxidizing agent is used in a 2-fold to 20-fold molar excess, particularly preferably in a 3-fold to 15-fold excess and very particularly preferably in a 4-fold to 10-fold excess, based on the tertiary amine or the nitrogen-containing aromatic heterocycle.

The selectivity of the reaction itself depends not only on the concentration of the reagents used but also on a number of other parameters, e.g. the temperature, the type of tertiary amine or nitrogen-containing aromatic heterocycle used or the residence time. Those skilled in the art will be able to adapt the various parameters to the particular oxidation reaction to give the desired amine oxide(s).

It is essential for the process according to the invention that the tertiary amines and/or nitrogen-containing aromatic heterocycles and oxidizing agents used are either themselves liquid or present in dissolved form. If they are not already themselves in liquid form, they therefore have to be dissolved in a suitable solvent before the process according to the invention is carried out. The solvents used are preferably halogenated solvents, particularly preferably dichloromethane, chloroform, 1,2-dichloroethane or 1,1,2,2-tetrachloroethane, linear, branched or cyclic paraffins, particularly preferably pentane, hexane, heptane, octane, cyclopentane, cycloheptane or cyclooctane, linear, branched or cyclic ethers, particularly preferably diethyl ether, methyl tertbutyl ether, tetrahydrofuran or dioxane, aromatic solvents, particularly preferably toluene, xylenes, ligroin or phenyl ether, N-containing heterocyclic solvents, particularly preferably pyridine or N-methylpyrrolidone, or mixtures of these solvents.

In the process according to the invention, the danger for man and the environment due to escaping chemicals is substantially reduced, thereby improving safety when handling hazardous substances. The oxidation of tertiary amines and nitrogen-containing aromatic heterocycles by the process according to the invention further affords better control of the reaction conditions, e.g. reaction time and reaction temperature, than is possible in the conventional processes.

Also, in the process according to the invention, the explosion hazard associated with very highly exothermic oxidations is markedly reduced. The temperature can be individually selected and kept constant in every volume element of the system. The course of the oxidation reactions in the process according to the invention can be regulated very rapidly and precisely, making it possible to obtain the amine oxides in very good and reproducible yields.

It is also particularly advantageous that the process according to the invention can be carried out continuously. This makes it more rapid and more cost-effective than conventional processes and any quantity of amine oxides can be prepared without great expenditure on measurement and regulation.

The invention is illustrated below by means of an Example. This Example serves solely to illustrate the invention and does not limit the general inventive idea.

### EXAMPLE

Oxidation of 1-[3-(4-amidinophenyl)-2-oxo-5-oxazolidinylmethyl]piperazin-4-acetic acid to 1-[3-(4-amidinophenyl)-2-oxo-5-oxazolidinylmethyl]-4-oxopiperazin-4-acetic Acid

1-[3-(4-Amidinophenyl)-2-oxo-5-oxazolidinylmethyl]piperazin-4-acetic acid was oxidized with m-chloroperbenzoic acid in a static micromixer (Technische Universität Ilmenau, Fakultät Maschinenbau, Dr.-Ing. Norbert Schwesinger, Postfach 100565, D-98684, Ilmenau) with external dimensions of 40 mm×25 mm×1 mm, which had a total of 11 mixing stages each with a volume of 0.125 μl. The total pressure loss was approx. 1000 Pa.

The static micromixer was connected via an outlet and an Omnifit medium pressure HPLC connector (Omnifit, Great Britain) to a Teflon capillary with an internal diameter of 0.49 mm and a length of 1.0 m. The reaction was carried out at 0° C., the static micromixer and the Teflon capillary being kept at this temperature in a thermostated jacketed vessel.

A 2 ml disposable injection syringe was filled with part of a solution of 25 mg (0.04 mmol) of 1-[3-(4-amidinophenyl)-2-oxo-5-oxazolidinylmethyl]piperazin-4-acetic acid in 5 ml of saturated sodium hydrogencarbonate solution and another 2 ml disposable injection syringe was filled with part of a solution of 38 mg (0.22 mmol) of m-chloroperbenzoic acid in 5 ml of saturated aqueous sodium hydrogencarbonate solution. The contents of both syringes were then transferred to the static micromixer by means of a metering pump (Harvard Apparatus Inc., Pump 22, South Natick, Mass., USA).

Before the reaction was carried out, the experimental set-up was calibrated in respect of the dependence of the residence time on the pump throughput. The residence time was adjusted to 15 seconds. The yield of the desired amine oxide was determined by means of a Merck Hitachi LaChrom HPLC instrument. 60% (of theory) of 1-[3-(4-amidinophenyl)-2-oxo-5-oxazolidinylmethyl]-4-oxopiperazin-4-acetic acid was obtained.

1. Process for the oxidation of tertiary amines and/or nitrogen-containing aromatic heterocycles to amine oxides, characterized in that at least one tertiary amine and/or at least one nitrogen-containing aromatic heterocycle, in liquid or dissolved form, is mixed with at least one oxidizing agent, in liquid or dissolved form, in at least one microreactor, the mixture is reacted for a certain residence time and the amine oxide formed is optionally isolated from the reaction mixture.

2. Process according to claim 1, characterized in that the microreactor is a miniaturized continuous reactor.

3. Process according to claim 1 or 2, characterized in that the microreactor is a static micromixer.

4. Process according to one of claims 1 to 3, characterized in that the microreactor is connected via an outlet to a capillary, preferably a capillary capable of being kept at a constant temperature.

5. Process according to one of claims 1 to 4, characterized in that the volume of the microreactor is ≦100 μl, preferably ≦50 μl.

6. Process according to one of claims 1 to 5, characterized in that the microreactor is capable of being kept at a constant temperature.

7. Process according to one of claims 1 to 6, characterized in that the microreactor has channels with a diameter of 10 to 1000 μm, preferably of 20 to 800 μm and particularly preferably of 30 to 400 μm.

8. Process according to one of claims 1 to 7, characterized in that the reaction mixture flows through the microreactor at a rate of 0.01 μl/min to 100 ml/min, preferably of 1 μl/min to 1 ml/min.

9. Process according to one of claims 1 to 8, characterized in that the residence time of the compounds used in the microreactor or, if appropriate, in the microreactor and the capillary is ≦15 hours, preferably ≦3 hours and particularly preferably ≦1 hour.

10. Process according to one of claims 1 to 9, characterized in that it is carried out at a temperature of −100 to +250° C., preferably of −78 to +150° C. and particularly preferably of 0 to +40° C.

11. Process according to one of claims 1 to 10, characterized in that the course of the reaction is monitored by chromatography, preferably by high performance liquid chromatography, and optionally regulated.

12. Process according to one of claims 1 to 11, characterized in that the amine oxide is isolated from the reaction mixture by extraction.

13. Process according to one of claims 1 to 12, characterized in that the oxidizing agent used is at least one oxidizing agent selected from inorganic and organic peroxides, hydrogen peroxide, mixtures of peroxo compounds with organic acids and/or inorganic acids and/or Lewis acids, organic per-acids, inorganic per-acids and dioxirans, or a mixture of at least two of these oxidizing agents.

14. Process according to claim 13, characterized in that the inorganic peroxide used is an ammonium peroxide, an alkali metal peroxide, preferably sodium peroxide, an ammonium persulfate, an alkali metal persulfate, an ammonium perborate, an alkali metal perborate, an ammonium percarbonate, an alkali metal percarbonate, an alkaline earth metal peroxide, zinc peroxide or a mixture of at least two of these peroxides.

15. Process according to claim 13 or 14, characterized in that the organic peroxide used is tert-butyl hydroperoxide, cumene hydroperoxide, menthyl hydroperoxide, 1-methylcyclohexane hydroperoxide or a mixture of at least two of these peroxides.

16. Process according to one of claims 13 to 15, characterized in that potassium peroxodisulfate with sulfuric acid is used as the peroxo compound with an inorganic acid, and hydrogen peroxide with boron trifluoride is used as the peroxo compound with a Lewis acid.

17. Process according to one of claims 13 to 16, characterized in that the organic per-acid used is peroxybenzoic acid, m-chloroperoxybenzoic acid, p-nitroperoxybenzoic acid, magnesium monoperoxyphthalic acid, peroxyacetic acid, peroxymaleic acid, peroxytrifluoroacetic acid or a mixture of at least two of these per-acids.

18. Process according to one of claims 1 to 17, characterized in that the tertiary amine used is an aliphatic, cycloaliphatic, aromatic or heteroaromatic tertiary amine, preferably trimethylamine and/or triethylamine.

19. Process according to one of claims 1 to 18, characterized in that the nitrogen-containing aromatic heterocycle used is a nitrogen-containing aromatic heterocycle with a ring size of 5 to 7 atoms, preferably 5 or 6 atoms, and is particularly preferably pyridine and/or pyrimidine and/or pyrazine.

20. Process according to one of claims 1 to 19, characterized in that the molar ratio of tertiary amine and/or nitrogen-containing aromatic heterocycle to oxidizing agent is equimolar or in that the oxidizing agent is used in a 2-fold to 20-fold molar excess, preferably in a 3-fold to 15-fold excess and particularly preferably in a 4-fold to 10-fold excess, based on the tertiary amine and/or the nitrogen-containing aromatic heterocycle.
